(19) United States
(12) Patent Application Publication
(10) Pub. No.: US 2007/0135780 A1
(43) Pub. Date: Jun. 14, 2007

(54) Surgical bag and morcellator system and method of use

(76) Inventor: Anthony C. Pagedas, Greendale, WI (US)

Correspondence Address: RYAN KROMHOLZ & MANION, S.C., POST OFFICE BOX 26618, MILWAUKEE, WI, 53226, US

(21) Appl. No.: 11/297,908

(22) Filed: Dec. 9, 2005

(51) Int. Cl. A61M 1/00

(52) U.S. Cl. 604/327

(57) ABSTRACT

A surgical bag adapted for electrical coupling to a tissue morcellator. The bag comprises a first layer comprising an electrically non-conductive material and a second layer comprising an electrically conductive material. Electrical communication between the second layer and the morcellator completes an electrical circuit and prevents movement of the morcellator or otherwise alerts the operator that contact between the morcellator and bag has occurred.

### FIELD OF THE INVENTION

The present invention generally relates to surgical equipment and specifically to tissue morcellators and surgical bags used in laparoscopic or similar types of surgery.

### BACKGROUND OF THE INVENTION

Laparoscopic tubes and sleeves with diameters on the order of 10 millimeters are inserted into a body cavity, although the diameter may increase or decrease depending upon the instrument used or the need. Various instruments and a video camera are typically directed through laparoscopic sleeves for performing and monitoring the surgical steps. The video camera, while working in a three dimensional environment, produces a two dimensional image for physician guidance during surgery.

A particular concern in laparoscopic surgery is the transporting of tissues and other masses that are cut away or retrieved during a surgery. While moving, manipulating, or cutting up a removed mass within the body cavity, pieces of infected or cancerous mass, blood, bile, and other liquids may escape into the body cavity and pose infection problems or other complications. These materials are desirably removed by placing them in a bag or similar enclosure within the body cavity before removal to minimize the risk of infection or other complications. It is important that the containment of the materials be accomplished as quickly as possible with minimal disturbance to the surgical site.

Laparoscopic retrieval instruments with membranes or bags have been designed in an attempt to avoid the complications associated with the removal of tissue during laparoscopic surgery. These instruments typically fall into two categories. The first category includes devices that have a bag coiled around an introducing rod that must be unfurled by various maneuvers. The second category includes devices that pop open the bag using a spring wire, or other mechanisms. Examples of the second category are disclosed in U.S. Pat. Nos. 6,059,793, 6,258,102, and 6,387,102, hereby incorporated by reference, which utilize a flexible rod slidably connected to a wand to create a reclosable pouch. When the rod bows but, the bag is opened. When the rod straightens, the bag is closed to form a seal.

Typically when a bag is used to contain the tissue or other material that is to be removed through the small incision, it is necessary to first use a morcellator to reduce the size of the tissue mass so that it can fit through the small incision. An example of a morcellator is found in U.S. Pat. No. 6,045,566, hereby incorporated by reference. Accordingly, the top portion of the bag is removed from the body cavity through one of the small incisions. The top portion of the bag is then opened so that the morcellator may be extended into the bag and into contact with the tissue or other material that is to be reduced in mass. While the use of these bags and morcellators are advantages during surgical procedures and reduce the necessary size of incisions needed for such procedures, a problem with these instruments is that specimen bags are often ruptured by the use of known morcellating instruments. A rupture in the bag may lead to inadvertent dispersal within the body cavity of the mass that is to be removed. Additionally, known morcellating instruments are so powerful that they can quickly and inadvertently cut through healthy tissue, bone, or even the operating table, which can lead to serious injuries.

For the foregoing reasons, there is a need for a surgical bag and morcellator system that allows for efficient reduction of the targeted tissue material by a morcellator but safeguards the bag against possible rupture or severing of the bag by the morcellator during use.

Additionally, there is a need for a surgical bag and morcellator systems with built in fail safe devices that cause the morcellator to automatically turn off if the morcellator begins to cut through the surgical bag.

### SUMMARY OF THE INVENTION

It is an object of this invention to provide a surgical bag system that allows for efficient reduction of the targeted tissue material by a morcellator but is safeguarded against rupture or severing by the morcellator during use.

It is also an object of this invention to provide a surgical bag system with a built in fail safe device that causes the morcellator to automatically turn off if the morcellator begins to cut through the surgical bag.

These and other objects of the invention are achieved by providing a surgical bag comprising a first layer comprising an electrically non-conductive material and a second layer comprising an electrically conductive material. The bag is electrically coupled to a tissue morcellator. Movement of the tissue morcellator is stopped in response to electrical contact between the morcellator and the electrically conductive layer of the bag. An electrical circuit communicates with the morcellator and the bag. The circuit comprises a controller and a power source. When the morcellator is in contact with the second layer, the circuit will communicate with the controller to prevent further movement of the morcellator and/or provide a signal to alert the user of that contact has been made with the second layer of the bag.

According to one aspect of the invention, the bag further comprises a third layer comprising an electrically non-conductive material. The second layer is positioned between the first and third layers. Other layers may be added to the bag, if desired.

### BRIEF DESCRIPTION OF THE DRAWINGS

FIG. 1 is a partially cut-away view of a tissue morcellator and a laparoscopic retrieval instrument carrying a surgical bag positioned within a body cavity for the processing and retrieval of a mass or other biological tissue.

FIG. 2 is a fragmentary partially cut-away close-up view of the morcellator and bag of FIG. 1.

FIG. 3 is a fragmentary cut-away close-up view illustrating an alternative embodiment of the surgical bag.

FIG. 4 is a schematic view illustrating an electrical circuit for a morcellator and surgical bag according to the present invention.

FIG. 5 is a diagrammatic view of an electrical circuit according to the present invention.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

FIG. 1 illustrates a tissue morcellator 10 and a laparoscopic retrieval instrument 12 within a body cavity 18. The retrieval instrument 12 further comprises a rod 14 having a handle 15 for easy manipulation and for carrying a surgical bag 16 within the body cavity 18. The instrument 12 and the surgical bag 16 assist in the processing and retrieval of a mass 20 or other biological tissue. The morcellator 10 comprises a handle 11 having a trigger 13 for controlling movement of the morcellator 10. Alternatively, the trigger 13 could comprise other means, such as a foot switch (not shown). The handle 11 is connected to a shaft 19 that terminates at an operating end 17. The morcellator 10 is used to cut-up the mass 20 so that it may be more easily removed from the body cavity 18. The morcellator 10 may be of any style-or type known in the art. Likewise, the retreival instrument 12 may also be of any style or type known in the art.

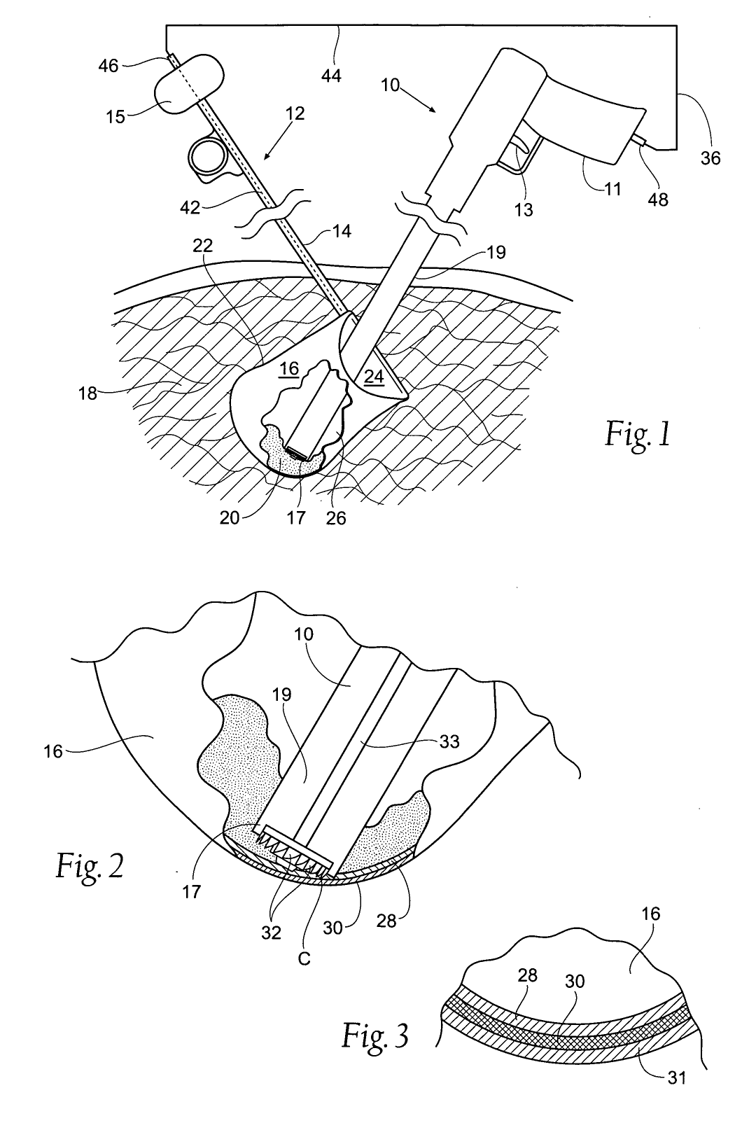

Referring to FIGS. 1 and 2, the bag 16 has a wall 22 defining an entryway 24 for surrounding an interior space 26. In a preferred embodiment, the bag wall 22 includes at least two layers, as seen in FIG. 2. An inner or first layer 28 of the bag 16 preferably is fabricated from a polymer or other biocompatible electrically non-conductive material, e.g., transparent plastics such as polyurethane, nylon, latex, flexible fabrics, or other similar materials. The preferred materials are impermeable to liquid or are liquid-resistant for containing infected liquids. A second or outer layer 30 is positioned over the first layer 28 and is preferably fabricated from a biocompatible electrically conductive material, e.g., surgical steel mesh, or a polymer impregnated with a conductive particle. Alternatively, the first layer 28 could be coated with a metallic or otherwise conductive material and the coating would be considered the second layer 30. As such, the second layer 30 should be considered broadly to include any arrangements of materials that include an electrically conductive material or materials. The preferred materials for both the non-conductive and conductive layers 28 and 30 preferably have some memory for tending to stay in a somewhat expanded state rather than tending to collapse, since this feature aids in the quick opening or unwrapping of the bag 16. The bag 16 can be disposable or reusable. The preferred materials for the bag 16 should also meet sterility and safety requirements for internal surgery. The first and second layers 28 and 30 may be fixedly coupled together, e.g., by lamination, coating, or other known coupling means.

As shown in FIG. 1, the bag 16 is opened. The surgeon then inserts the morcellator 10 into the interior space 26 of the bag 16 for further manipulation of the mass 20, for example, to cut a large mass 20 into pieces. The non-conductive layer 28 prevents contact between the conductive layer 30 and the morcellator 10 and the morcellator 10 operates normally.

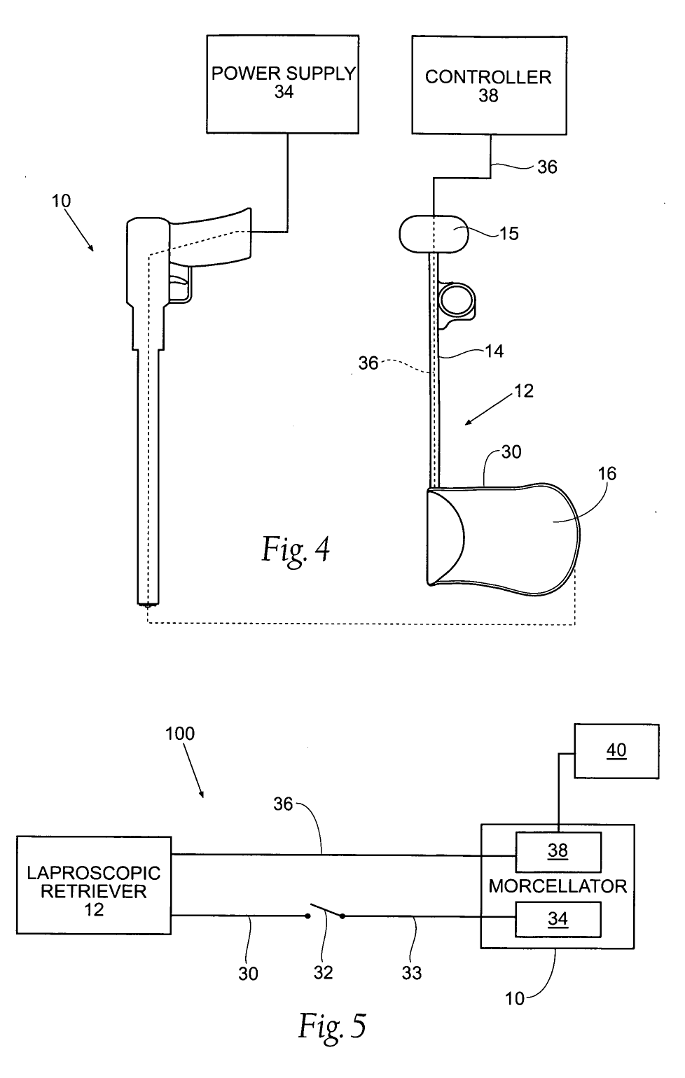

In FIG. 2, the operating end 17 of the morcellator 10 is shown further comprising a blade or blades 32. The blade 32 is connected to an axle 33, which provides movement for the blade 32. The blade 32 comprises a conductive material, as preferably does the axle 33. Alternatively, the axle 33 may be formed of a non-conductive material, provided that there is an electrical wire, conduit or other means connecting the blades 32 to the power source of the morcellator 10 (see FIG. 4). Preferably, the shaft 19 is made of a non-conductive material and acts as an insulator for the axle 33. As the surgeon or operator is cutting up the mass 20 and moving the morcellator 10 around within the bag 16, the operator may inadvertently cut through or tear the first layer 28 of the bag 16. In response to tearing or cutting through of the non-conductive layer 28 and contact between the blade 32 and the conductive layer 30, shown as point C, a circuit is completed, and further movement of the morcellator is prevented. Alternatively, an audio and/or visual signal may be activated when the blade 32 comes into contact with the conductive layer. Such safety features safeguard against the morcellator 10 penetrating the surgical bag 16 completely and inadvertently damaging the surrounding tissue or body cavity and/or leaking the contents of the bag 16. If the second layer 30 had been possibly severed, the use of the third layer 31 will further protect against unwanted dissemination of contents within the bag 16.

It is contemplated that additional layers may be provided as desired for extra protection. For example, as shown in FIG. 3, a non-conductive third layer 31 may be provided over the second layer 30. The third layer 31 may be formed of the same material as the first layer 28, or any other suitable biocompatible material. If desired, the third layer 31 may be fixedly coupled to the second layer, e.g., by lamination. The use of a third layer 31 may further prevent the leaking of the contents of the bag 16 into the surrounding area.

As shown in FIGS. 1, 2, and 3 the layers 28 and 30 are adjacent to one another. While such an arrangement is preferable, it has been contemplated that other layers of material may be located between the layers 28 and 30. It is understood that the scope of the present invention will include such arrangements.

FIGS. 4 and 5 show and describe a possible circuit arrangement used in the present invention. The morcellator is connected to a power supply 34. The power supply 34 may be an external source or may be located within the morcellator 10. Preferably, a lead wire 36 is connected to the second layer 30 of the bag. The lead wire 36 preferably follows the rod 14 of the instrument 12, either internally of the rod 14 or along the outside the rod 14. While it is not necessary for the wire 36 to be in close proximity to the rod 14, the arrangement eliminates potential problems from the wire 36 becoming tangled or interfering with the surgical procedure and allows for easier insertion into a body cavity. The wire 36 continues onward and is connected to a controller 38, which will provide the necessary means for preventing further movement of the blades 32 of the morcellator 10. As stated with the power source 34, the controller 38 may be located internally or externally of the morcellator 10. The wire 36 should be considered broadly as any electrical conduit that connects the bag 16 to the controller 38.

Referring to FIG. 5, a schematic diagram of a possible circuit 100 used according to the present invention is shown. The circuit 100 passes from the power source 34, through the axle 33 and to the blade or blades 32, which may come into contact with the conductive layer 30. As shown, the blades 32 are not in contact with the conductive layer 30, and the morcellator operates normally. The conductive layer is connected to the lead wire 36 housed within the instrument 12. The lead wire 36 continues onward to the controller 38.

Alternatively, or in connection with halting movement and operation of the morcellator 10, the controller 38 may be connected to a warning device 40 to alert the surgeon that electrical contact with the layer 30 has occurred. The warning device 40 may be an audio signal (e.g., a buzzer) or a visual signal (e.g., a warning light).

The controller 38 is preferably a standard general purpose I/O device or microprocessor. Other arrangements, such as fuses or breakers may be used, but the use of the microprocessor allows for quicker response times when the blades 32 come into contact with the layer 30. Because blades on a typical morcellator move or rotate at approximately 2,000-6,000 rpm, it is necessary to have a quick response when the morcellator cuts through a containment bag. Consequently, the use of a microprocessor allows response to the contact within fractions of a second, before the containment bag is completely severed. Likewise, depending on the composition of the layers 28, 30 of the bag, the response of the controller 38 may be programmed for different levels of sensitivity

The power control 34 may be of any suitable arrangement that typically is used within a surgical or operating environment. One example for the power control 34 would be a 5V battery located within the morcellator 10. However, as previously stated any suitable internal or external power source may be used, such as connection to an electrical outlet or wall socket (not shown).

Referring again to FIG. 1, the lead wire 36 may be designed as comprising separate sections. An internal section 42 may be coupled to an external section 44 by a plug or other similar socket 46. Likewise, the external section 44 may be further coupled to the morcellator 10 by a second plug or socket 48. Such an arrangement would make it easy to disassemble and store the morcellator 10 and instrument 12 when not in use.

As shown and discussed, the blades 32 on the morcellator 10 provide the potential electrical connection between the bag 16 and the morcellator 10. It is understood that a different portion of the morcellator 10 could be used to complete the circuit 100. For instance, the operating end 17 that surrounds the blades 32 may be electrically conductive to provide the necessary bridge to complete the circuit 100. Provided that a circuit is completed when contact is made with the outer layer 30 and a portion of the morcellator 10, the design should fall under the scope of the present invention.

The present invention provides a surgical bag that will minimize potential hazardous problems with removal of masses from a body cavity. While the present invention has been discussed for use in conjunction with a morcellator, it is understood that the bag of the present invention could be used with other surgical instruments and procedures where it is preferable to contain a mass within a specified area, especially if the surgical instruments may cut through or puncture the bag.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I claim:

1. A surgical bag comprising:
a first layer comprising an electrically non-conductive material, and
a second layer comprising an electrically conductive material.

2. The surgical bag of claim 1
wherein the first and second layers are adjacent to one another.

3. The surgical bag of claim 1
wherein the first layer is laminated to the second layer.

4. The surgical bag of claim 1
wherein the second layer comprises a conductive coating located on said first layer.

5. The surgical bag according to claim 1, further comprising:
a third layer comprising an electrically non-conductive material, and
wherein the second layer is positioned between the first and third layers.

6. The surgical bag of claim 1 further comprising means for electrically coupling said bag to a control device, said control device selectably controlling movement of a surgical instrument.

7. A surgical system comprising:
a tissue morcellator having an electrically conductive area, and
a surgical bag comprising an electrically non-conductive first layer and an electrically conductive second layer; and
a control device, said bag electrically connected to said control device.

8. The surgical system of claim 7
wherein the tissue morcellator is prevented from movement by said control device in response to contact between said electrically conductive area of the morcellator and said second layer of said bag.

9. The surgical system of claim 8
wherein the first and second layers are adjacent to one another.

10. The surgical system of claim 8
wherein the first layer is laminated to the second layer.

11. The surgical system of claim 8.
wherein the second layer comprises a conductive coating located on said first layer.

12. The surgical system according to claim 8, further comprising:
a third layer comprising an electrically non-conductive material, and
wherein the second layer is positioned between the first and third layers.

13. The surgical system according to claim 7 further comprising an alarm, said alarm connected to said control device, said alarm signaled when contact is made between said morcellator and said second layer of said bag.

14. A surgical assembly comprising:
a surgical instrument having an electrically conductive area;
a power source for operation of said surgical instrument, said power source in electrical communication with said electrically conductive area;
a surgical bag comprising an electrically non-conductive first layer and an electrically conductive second layer;
a control device; and
a control circuit connecting said electrically conductive area of said instrument, said conductive layer of said bag, and said control device.

15. The assembly according to claim 14 further comprising means for preventing operation of said surgical instrument when said circuit is completed.

16. The assembly according to claim 14 wherein said means for preventing operation operation of said surgical instrument further comprises contact between said electrically conductive area and said second layer of said bag.

17. The assembly according to claim 14 further comprising an alarm, said alarm activated when said circuit is completed.

18. The assembly according to claim 14 wherein said surgical bag is releasably connected to said control device.

19. The assembly according to claim 14 further comprising a lead wire connected, said lead wire connecting said surgical bag to said control circuit.

20. The assembly according to claim 14 wherein said control device is located within said surgical instrument.
